(19) United States
(12) Patent Application Publication
(10) Pub. No.: US 2013/0078314 A1
(43) Pub. Date: Mar. 28, 2013

(54) TRANSDERMAL METHOD OF DELIVERING NUTRIENTS INTO HUMAN BODY

(71) Applicant: Peter Chan, Winston Hills (AU)

(72) Inventor: Peter Chan, Winston Hills (AU)

(21) Appl. No.: 13/423,084

(22) Filed: Mar. 16, 2012

Related U.S. Application Data
(60) Provisional application No. 61/538,795, filed on Sep. 24, 2011.

(51) Int. Cl. A61K 35/12; A61K 35/34; A61M 35/00; A61K 36/00; A61P 3/02; A61K 35/407; A61K 35/30

(52) U.S. Cl. 424/553; 424/520; 424/569; 424/570; 424/725; 604/290

(57) ABSTRACT

The present invention is about a transdermal method that deliveries nutrients from fresh animal organs to the systemic circulation of the human body by applying the juice or chopped pieces of the fresh animal organs on the skin of the human body and allowing the nutrients to absorb through the layers of the exposed skin. The present invention aims to preserve the Yin and Yang energies naturally present in fresh organs and other foods that are lost in cooking and processing of foods. The present invention uses the full skin area transdermal delivery method by soaking the whole body in the juice of the fresh animal organs as well as the partial skin area transdermal delivery method by exposing certain parts of the human skin area to the fresh juices or chopped fresh animal organs and allowing the nutrients to enter the systemic circulation of the human body.

### REFERENCES CITED

US PATENT DOCUMENTS

| | | Filing | Issue | | |
|---|---|---|---|---|---|
| Pat. No. | | date | date | Original Assignee | Title |
| U.S. Pat. No. | 5,055,296 | Aug. 4, 1988 | Oct. 8, 1991 | | Method of treating chronic |
| | | | | | fatigue syndrome |
| U.S. Pat. No. | 5,284,664 | Feb. 5, 1992 | Feb. 8, 1994 | Kremers-Urban Company | Method of treating the |
| | | | | | symptoms of Alzheimer's |
| | | | | | disease |
| U.S. Pat. No. | 5,316,775 | Apr. 19, 1993 | May. 31, 1994 | Kremers-Urban Company | Method of treating hepatitis B |
| | | | | | infection |
| U.S. Pat. No. | 6,350,472 | Dec. 14, 1998 | Feb. 26, 2002 | Steinbach, Pylant, and | Method of treating HIV |
| | | | | Hermann, L.L.C. | infection with transdermal gel |
| | | | | | containing mammalian liver |
| | | | | | extract |
| U.S. Pat. No. | 5,334,395 | Aug. 24, 1992 | Aug. 2, 1994 | Kremers-Urban Company | Method of treating an epstein- |
| | | | | | barr viral infection |
| U.S. Pat. No. | 4,254,103 | Jun. 8, 1979 | Mar. 3, 1981 | Institul de Cercetari | Hepatoprotector Factor (HF) |
| | | | | Chimicofarmaceutice | and method of treatment |
| U.S. Pat. No. | 4,552,762 | Dec. 8, 1983 | Nov. 12, 1985 | Juanita Nepon | Methods of Preparing an |
| | | | | | Arthritis Composition |
| U.S. Pat. No. | 4,436,723 | Sep. 24, 1980 | Mar. 13, 1984 | Juanita Nepon | Arthritis composition of |
| | | | | | matter, and methods of |
| | | | | | preparing and utilizing same |
| U.S. Pat. No. | 3,637,641 | Jan. 16, 1970 | Jan. 25, 1972 | Wolfgang Huber | Method of Treating Post- |
| | | | | | Traumatic Arthritis |

### OTHER PUBLICATIONS

Ablashi, V., Berneman, N., Lawyer, C., Kramarsky. B., Ferguson, M., & Komaroff, L., (1994) Antiviral vitro of Kutapressin against human herpes virus-6. In Vivo 1994; 8:581-86
Carey, C., (2005), American Scientists, Facts on File Library of American History, page 251, ISBN-13: 978-0816054992
Wiseman, N. & Ellis, A. (1997), Fundamentals of Chinese Medicine, 1997, Paradigm Publications, Brookline, Massachusetts, ISBN:0-912111-44-5
American Cancer Society (2009), American Cancer Society's complementary and alternative cancer methods handbook, American Cancer Society, Second Edition, 2009.

This application claims the benefit of provisional patent application U.S. Ser. No. 61/538,795 filed 24 Sep. 2011 by the present inventor.

The present invention is about a transdermal method that deliveries nutrients from raw and fresh (or unprocessed) animal organs such as beef liver to the systemic circulation of the human body by applying the juice or chopped pieces of the raw and fresh animal organs on the skin of the human body and allowing the nutrients to absorb through the layers of the exposed skin. The aim of the present invention is to preserve the Yin and Yang energies naturally present in raw and fresh organs and other foods that are lost in cooking and processing of foods. The present invention uses the full skin area transdermal delivery method by soaking the whole body in the juice of the fresh animal organs as well as the partial skin area transdermal delivery method by exposing certain parts of the human skin area to the fresh juices or chopped fresh animal organs and allowing the nutrients to enter the systemic circulation of the human body. When the nutrients enter the human body via the exposed skin, unlike existing transdermal methods, the present transdermal method maintains the following unique characteristics: (1) It allows nutrients from animal organs to maintain the attribute of being (a) “holistic” and (b) “fresh”. (2) It utilizes all area of human skin providing maximum opportunity for the human skin to absorb of nutrients from animal organs into human body.

### 1. FIELD OF THE INVENTION

The present invention relates generally to the delivery of nutrients into the human body. More particularly, the invention relates to the transdermal delivery of nutrients from raw, fresh, unprocessed animal organs into the human body.

### 2. BACKGROUND

Holistic approach (Yin and Yang Theory): The theory of Yin and Yang remains the cornerstone of Traditional Chinese Medicine (TCM) and fengshui practices today as it was when conceived back in the Yin Dynasty (about 1400-1100 BC). Yin and Yang exist in pairs and represent opposite principles in nature—the moon and the sun, female and male, give and take, left and right hemispheres of the brain, etc. All things in nature exist harmoniously only when Yin and Yang energies are precisely balanced.

Human health, from a Yin and Yang perspective, corresponds to a balanced choice of diet. Optimum health is achieved by balancing Yin and Yang and as quoted from Wiseman & Ellis (1997), “diseases arise when there is in-equilibrium of Yin and Yang inside the body”. By its very nature, unprocessed whole food is balanced. The concept of consuming whole foods (i.e. natural and unprocessed food) is an application of the Yin Yang theory.

In keeping with the holistic approach and the Yin and Yang philosophy, the present invention is based on the consumption and utilization of fresh unprocessed whole foods which by their very nature are completely in balance between Yin and Yang. Examples of unprocessed whole foods include, brown rice, brown sugar, whole fish including the head, eyes and intestines which are normally discarded, but deemed beneficial in preventing atherosclerosis and other diseases. More particularly, the present invention aims to deliver essential nutrients to the human body primarily through transdermal delivery to maximize the bioavailability of nutrients from food without their destruction through cooking and other means of processing.

To achieve these goals, the present invention uses a food source such as the animal liver which has a high concentration of essential nutrients and enzymes. Beef or pork liver extracts has been successfully used to treat a multitude of health issues. Examples of treatment of such issues includes but not limited to: the treatment of Chronic Fatigue Syndrome (U.S. Pat. No. 5,055,296); Alzheimer's Disease (U.S. Pat. No. 5,284,664); Hepatitis B infections (U.S. Pat. No. 5,316,775); HIV infections (U.S. Pat. No. 6,350,472); Epstein-Barr viral infections (U.S. Pat. No. 5,334,395); Anemia (Carey, 2005); Cirrhosis of the Liver and Viral Hepatitis Infections (U.S. Pat. No. 4,254,103); Human Herpes Virus Infections (Ablashi et al, 2004) and various forms of arthritis (U.S. Pat. No. 4,552,762; U.S. Pat. No. 4,436,723; U.S. Pat. No. 3,637,641). Treatment methods include the use of transdermal, intramuscular or oral delivery. Transdermal delivery method involves the application of transdermal patches or gel on an area of the human skin. Animal liver in tablet or powder form are also orally consume by athletes to enhance their athletic performance. These tablets or powder are available in sport stores or online stores as sport supplements.

As powerful as they are in their capability to treat (or help keep away from) a wide array of health issues, beef or pork livers, by nature is no exception in following the rule of Yin and Yang. Raw and fresh beef or pork livers come with an excellent balance of Yin and Yang energies. However, when beef or pork liver are treated or altered by way of cooking (or heating), processing, centrifuging, chemically-treating or preserving (usually to prolong its shelf life), it kills or discards many valuable nutrients and enzymes and along the way diminished the all important attribute of being “holistic”. To maintain a healthy life style, help keep away from harmful diseases and maintain peak performance human body needs to be well nourished and ecologically balanced within. Feeding a variety of powerful nutrients from raw and fresh animal organ such as beef or pork livers in a “holistic” manner helps maintain this all important balance. The method introduces in the present invention is about delivering the multitude of valuable (health or sport wise) nutrients naturally available within animal organ such as beef or pork livers into the human body, without compromising the lost of the attribute of being “holistic” and “fresh”.

There are a few shortcomings associated with transdermal as well as non-transdermal delivery methods known in the art: heating, cooking or extraction of certain nutrients destroys or isolates many nutrients and some enzymes within the animal organ; one can orally consume a limited amount of animal organs at one time and any amount above that limit or frequent consumption of a particular animal organs can make the consumer sick; for some individual, especially older people or people with dis-functional digestive systems (e.g. people with irritable bowel syndrome or Crohn's disease), the capability of digesting and/or absorbing nutrients can greatly diminish and orally consuming beyond certain amount of animal organs can be intolerable and make the consumer sick; when animal organs are centrifuged, heated or chemically treated to extract certain nutrients the attribute of being “holistic” and “freshness” diminish; and when it comes to transporting nutrient from animal organ to human body, transdermal methods known in the art utilize only a small portion of the skin area relative to the full skin area of body—under-utilizing its full absorbing potential.

Against the fore mentioned backgrounds, prior arts and associated shortcomings, the inventor have endeavored to devise a method to overcome the shortcomings poised by the transdermal delivery methods known in the art.

### 3. SUMMARY OF THE INVENTION

Human skin is the largest organ of human body and presents the largest area when it comes to absorbing nutrients. In present transdermal method, raw and fresh animal organ is transformed into a liquid (or juice) form using low intensity processing methods and the resulting animal organ in liquid (or juice) form is then applied (rubbed and massaged) onto all or most area of human skin area allowing maximum chance for the body to absorb nutrients from the animal organ. Transforming animal organ into liquid (or juice) form facilitates the transdermal delivery. Example of low intensity processing methods includes but not limited to, grinding animal organ using electric blender at a minimal speed and/or intermittently to maintain minimal temperature, preventing nutrients within from being destroyed due to overheating.

The present invention is about a “TRANSDERMAL METHOD OF DELIVERING NUTRIENTS INTO HUMAN BODY”. However, the present invention overcomes shortcomings associated with transdermal as well as non-transdermal methods known in the arts in one or more of the following ways: (1) the use of raw and fresh animal organs helps (a) preserves the Yin and Yang energies naturally present in the animal organs and maintains the attributes and principles of holism because no cooking or heating, extraction of nutrients by way of centrifuging nor chemical and/or preservative assisted extraction is involved; (b) maintains the attribute of being “fresh”; and (c) negate any adverse effect due to the use of preservatives or chemicals in processing animal organs; (2) the use of human skin as a mean to transporting nutrient from animal organ to human body addresses the issues related to oral consumption which includes intolerable digestive system due to dis-functional digestive systems in some individuals or orally consumption of animal organ beyond certain amount; and (3) the use of all or most area of human skin area as a mean to transport nutrient from animal organ to human body allows the present invention to maximize the full potential of the skin absorption capability.

In the prior transdermal delivery methods, animal organs such as beef or pork livers are processed (heated, centrifuged or chemically treated) one way or the other before applying onto a particular skin area of human body. The use of unprocessed (raw and fresh) animal organs such as beef or pork livers in the present invention is uncommon and unique. The application of unprocessed (raw and fresh) animal organs such as beef or pork livers on to all skin area of the human body in the present invention is also uncommon and unique.

In an exemplary embodiment of the present invention, an animal organ such as raw, fresh beef (cow or calf) liver is macerated and ground using an electric blender into the consistency of a juice. When grinding a minimal speed is observed to prevent the destruction of nutrients within animal organ from over heating. The juice is then applied all over the surface skin (epidermis) of the body by rubbing and massaging it into the skin and leaving it on for a period of time. This allows for the nutrients in the animal organ juice to traverse through the pores of the surface skin epidermal layer into the inner dermal layer and enter the systemic circulation through the blood vessels in the dermal layer of the skin. This embodiment of the invention uses the full skin area transdermal delivery method of distributing the nutrients in the animal organ into the human body.

Yet another exemplary embodiment of the invention uses the partial skin area delivery method for delivering the nutrients in the animal organ into the systemic circulation of the human body. In this embodiment of the invention, the raw, fresh animal organ is chopped up into small pieces, placed on an area of the exposed skin surface and taped using an adhesive water proof tape. In this embodiment, the absorption of nutrients from the animal organ is confined to the area of skin in close contact with the pieces of animal organ.

In a related embodiment of the invention, the raw, fresh animal organ is either cut or macerated into small pieces or prepared in juice form in an electric blender and placed in a container with an open top which is inverted on to an area of skin and the container is held in place with tapes. In this embodiment, the nutrients from the animal organ are absorbed into the skin through the area of skin exposed to the animal organ juice or the macerated pieces placed in the container.

In another related embodiment of the invention using the partial skin delivery method, the raw fresh animal organ is either chopped into pieces, macerated or blended to form a juice using an electric blender and placed into water proof gloves, stockings, or a boot that is worn by the individual and held in place with elastic bands, strong cord or other means to prevent the product from leaking and spilling out.

In another related embodiment of the invention, the macerated or cut raw, animal organ is placed in a rigid or flexible waterproof cylinder with two open ends and worn over the forearm, legs, around the chest or belly to allow the nutrients from the animal organ to be transported through the skin into the systemic circulation of the body.

In another yet related embodiment of the invention using the partial skin delivery method, the raw fresh animal organ is either chopped into pieces, macerated or blended to form a juice using an electric blender and placed into a water proof cap that is worn by the individual. With working principle resembling to that of a swimming or shower cap, the water proof cap will have a tight rim tight to prevent the product within from leaking and spilling out. In this embodiment, the nutrients from the animal organ are delivering into the human body through the area of scalp skin exposed to the animal organ juice or the macerated pieces placed in the container.

In another related embodiment of both the full skin delivery method and the partial skin delivery method, the raw, fresh beef or pork liver or heart or brain is the prefer raw, fresh animal organ.

In an alternate embodiment of both the full skin delivery method and the partial skin delivery method raw, fresh animal organ can be replaced with herbs or spices such as, Chinese five spice mix, Indian curry masala etc. In these embodiments of the invention, juices from the various animal organs and/or various herbs or spices can be combined for delivery of their nutrients.

Embodiments of the invention thus described may also be used for delivery of nutrients to other animals such as for instance, household pets, such as dogs and cats as well as other domestic farm animals, or other animals in general.

In this summary of the invention and in the specification in general, the various references to “exemplary embodiment”, “related embodiments,” “yet another embodiment,” or “alternate embodiment” do not all necessarily refer to the same embodiment (s). Rather, these references in general mean that a particular feature, structure, or characteristic described in connection with an embodiment is included in at least some embodiments, but not necessarily all embodiments of the invention. The features and advantages of the embodiments of the present invention as described in the summary of the invention will be further appreciated and will become obvious to one skilled in the art when viewed in conjunction with the accompanying drawings, detailed description of the invention and the appended claims.

### 4. DETAILED DESCRIPTION OF THE INVENTION

In prior transdermal methods, processed organs such as liver in the form of gel or patch or other forms are used to apply onto a certain area of the human skin such as the forearm. When organs such as liver are processed, i.e. heated or centrifuged or chemically isolated it removes two valuable attributes from the organ: (1) the attribute of being holistic and (2) the attribute of being fresh. When organs such as liver are processed some of the nutrients are destroyed or isolated. From a Yin and Yang prospective, processed organ equals ecological imbalance organ.

A major aspect of Traditional Chinese medicine, as quoted from American Cancer Society (2009), is about, “focusing on restoring a balance of energy, body, and spirit to maintain health rather than treating a particular disease or medical condition”. In line with the Traditional Chinese Medicine, the present invention concentrates on the holistically refueling of Yin and Yang energies to the human body. It does not focus on treating any particular disease or medical condition.

The present invention has a few advantages over the prior art in transdermally delivering of nutrients from animal organs such as liver into the human body: (1) the use of fresh or raw organs such as liver allows the present invention to maintain the holistic balance within the organ when the nutrients within are transdermally absorbed into the human body; (2) the use of fresh or raw organs such as liver allows the present invention to maintain the attribute of being fresh when the nutrients within are transdermally absorbed into the human body; and (3) the application of fresh or raw organs such as liver over the whole body skin area in the manner akin to having a liver bath allows the present invention to maximize the absorption of nutrient into the human body.

Referring now to the figures, more particularly FIG. 1, an embodiment of the invention 1 using the partial skin delivery method is shown where the chopped or macerated fresh beef liver 2 is applied on the surface of the skin and retained in place by strips of adhesive bandage tapes 3. In this embodiment, the liver is left in contact with the skin for a period of time so that the nutrients from the liver traverse through the epidermis of the skin into the dermis layer and eventually enter the systemic circulation of the body.

FIG. 2 is a perspective view depicting another embodiment of the partial skin delivery method of the present invention 1. In this embodiment, a container 4 is used to hold the chopped and macerated beef liver or fresh food from other sources and the open end of the container 4 with the fresh beef liver or other fresh food is permitted to come in contact with the skin, with the container held in place by adhesive bandage or other tapes 3.

FIG. 3 shows the perspective view of another embodiment of the invention 1 using the partial skin delivery method where a waterproof glove 5 holds the liver juices or macerated pieces and is tied to the wrist of the hand 7 with a sturdy cord or string 6 or in the alternative a elastic band. In this embodiment, the liver juices or pieces are allowed to remain in contact with the skin on the palm, fingers and back of the hand for a period of time to allow the nutrients in the juice to enter the skin layers and into the systemic circulation.

In FIG. 4 which shows another perspective view of the present invention 1 using the partial skin delivery method, the fresh liver or other food juice or chopped and macerated pieces of the liver or other fresh food are placed within the waterproof boots 8 and the top of the boots tied to the legs 9 using the cord 6 or in the alternative a elastic band to prevent the juices or pieces from leaking out of the boot 8. In this embodiment, the liver or other juices or chopped pieces are allowed to remain in contact with the skin on the feet for a period of time so that the nutrients from the fresh liver or food can enter the skin layers and into the systemic circulation of the body.

FIG. 5 is another embodiment of the invention 1 where a cylinder 10 with two open ends is inserted over the forearm, leg, around the chest, or belly of the human body and the chopped or macerated pieces or juice of the liver or other fresh food allowed to remain in contact with the skin for a period of time for the nutrients to enter the skin layers and eventually into the bodies systemic circulation. In this embodiment, the cylinder is closed on both sides after inserting the body part and tied in place with cord 6 or in the alternative a elastic band to prevent the contents from leaking out.

FIG. 6 shows two perspective views of another embodiment of the invention 1 using the partial skin delivery method where a waterproof cap 11 or 12 holding the liver juices or macerated pieces is worn by an individual. The rim of the waterproof cap 11 or 12 is tighten to prevent the product within from leaking and spilling out. The working principle of the waterproof cap 11 resembles that of the a swimming cap whereby the rim of the waterproof cap 11 is self-tightening by its own elasticity. The working principle of the waterproof cap 12 resembles that of the a shower cap whereby the rim of the of the waterproof cap 12 is tighten with a sturdy cord or string 6 or in the alternative a elastic band.

The foregoing description of the present invention through its various embodiments should not be construed to limit the scope of the invention. It should be understood and obvious to one skilled in the art that the embodiments of the invention thus described may be further modified without departing from the spirit and scope of the invention as set forth in the claims.

### 5. BRIEF DESCRIPTION OF THE DRAWINGS

FIG. 1 is a perspective view of an embodiment of the invention using the method of partial skin transdermal delivery of nutrients.

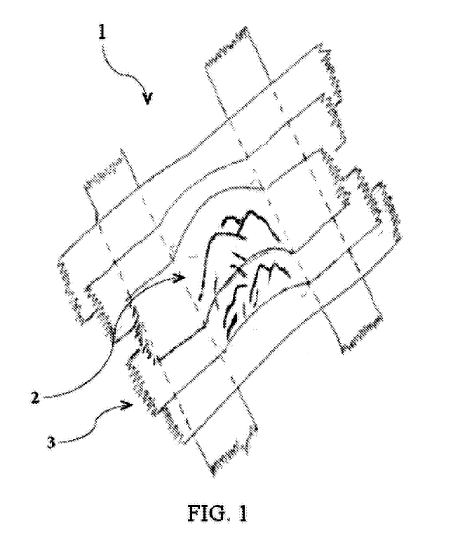

FIG. 2 is a perspective view of another embodiment of the invention using the partial skin transdermal delivery of nutrients method.

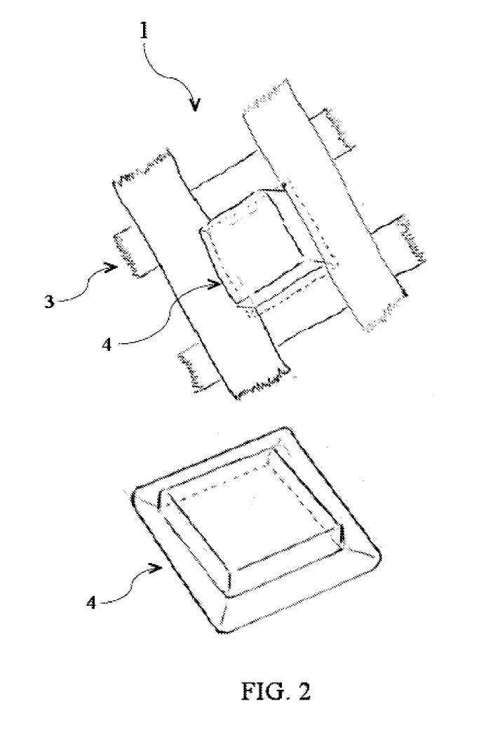

FIG. 3 is a perspective view of the embodiment of the invention using a waterproof glove to contain the fresh juice of beef liver or nutrient rich juices from other similar sources.

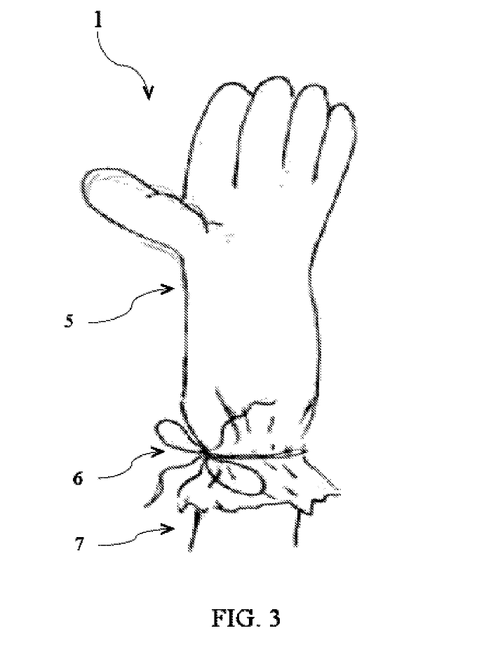

FIG. 4 is a perspective view of yet another embodiment of the invention using a waterproof boot to contain the fresh nutrient rich juices from various sources.

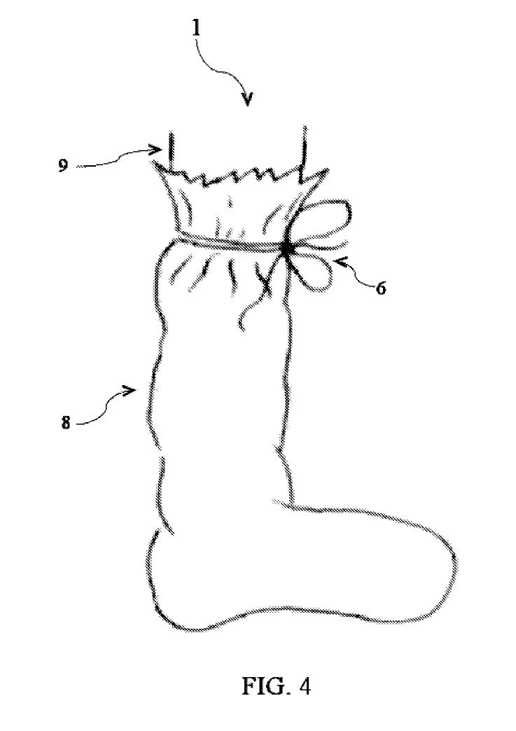

FIG. 5 is a perspective view depicting the use of a cylinder to hold the nutrient rich fresh juice around the forearm, leg, chest, or belly of the body.

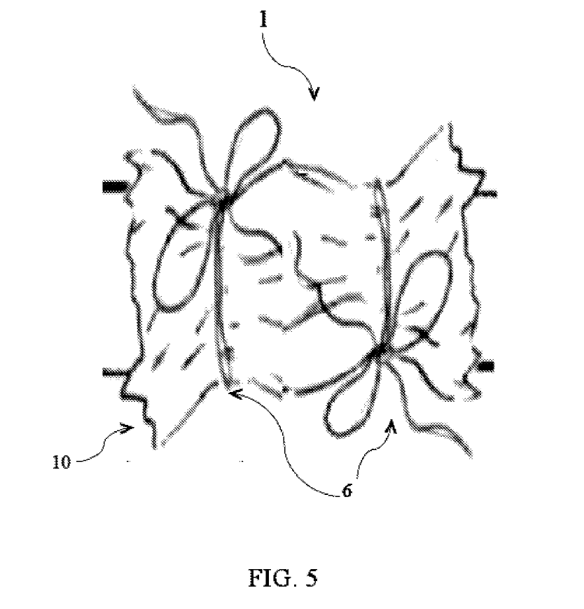

FIG. 6 is two perspective views of the embodiment of the invention using waterproof caps to contain the fresh juice of beef liver or nutrient rich juices from other similar sources.

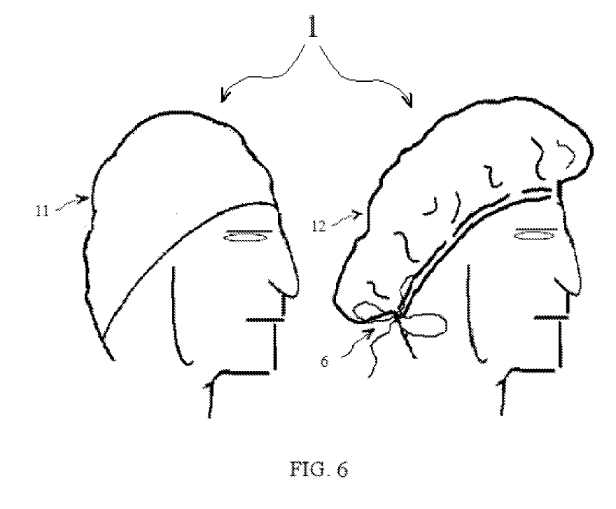

What is claims is:

1. A method for transdermal delivery of nutrients into the human or animal body, the method comprising: preparing a juice of raw, fresh organ meat with no heat and/or minimal processing to preserve the nutrients in their natural state and allowing the said raw, fresh organ meat juice to come into contact with the skin of the whole body by soaking the said whole body in the said raw, fresh organ meat juice; and chopping and macerating the raw, fresh organ meat with no heat and/or minimal processing to preserve the nutrients in their natural state and allowing the said chopped and macerated raw, fresh organ meat to come into contact with the skin on parts of the body.

2. The method of claim 1 wherein the method for transdermal delivery of nutrients by soaking the whole body in the organ meat juice delivers nutrients from the raw, fresh organ meat to the body by the full skin area transdermal delivery method.

3. The method of claim 1 wherein the method of allowing the chopped and macerated raw, fresh organ meat to come into contact with parts of the body delivers nutrients from the organ to the body by the partial skin area transdermal delivery method.

4. The method of claim 3 wherein the partial skin area transdermal delivery method is accomplished by placing the chopped and macerated raw, fresh organ meat on a partial skin area of the body, holding it in place using adhesive bandage or other tapes and leaving it in place for a period of time so that the nutrients from the organ meat enter the systemic circulation of the body through the outer epidermis and inner dermis skin layers.

5. The method of claim 3 wherein the partial skin area transdermal delivery method is accomplished by placing the chopped and macerated raw, fresh organ meat or fresh organ meat juice on the partial skin area of the body by placing the chopped meat or juice in a container with the open end of the container in contact with the skin and holding the container in place using adhesive bandage or other tape for a period of time so that the nutrients from the organ meat enter the systemic circulation of the body through the outer epidermis and inner dermis skin layers.

6. The method of claim 3 wherein the partial skin area transdermal delivery method is accomplished by placing the chopped and macerated raw, fresh organ meat in a waterproof glove worn over the hand and tied with a cord or elastic band to prevent leakage, leaving it in place for a period of time so that the nutrients from the organ meat enter the systemic circulation of the body through the outer epidermis and inner dermis skin layers in the hand.

7. The method of claim 3 wherein the partial skin area transdermal delivery method is accomplished by placing the chopped and macerated raw, fresh organ meat in a waterproof boot or stockings worn over the foot and tied with a cord or elastic band to prevent leakage, leaving it in place for a period of time so that the nutrients from the organ meat enter the systemic circulation of the body through the outer epidermis and inner dermis skin layers in the foot.

8. The method of claim 3 wherein the partial skin area transdermal delivery method is accomplished by placing the chopped and macerated raw, fresh organ meat in a waterproof, flexible cylinder worn over the forearm, leg, around the chest, belly or any part of the body and tied with a cord or elastic band at both ends of the cylinder to prevent leakage, leaving it in place for a period of time so that the nutrients from the organ meat enter the systemic circulation of the body through the outer epidermis and inner dermis skin layers in those parts of the body.

9. The method of claim 3 wherein the partial skin area transdermal delivery method is accomplished by placing the chopped and macerated raw, fresh organ meat in a waterproof cap worn over the head with the rim of the waterproof cap tightly seal by its own elasticity or by using a cord or elastic band to prevent leakage and leaving the chopped and macerated raw, fresh organ meat in place for a period of time so that the nutrients from the organ meat enter the systemic circulation of the body through the outer epidermis and inner dermis skin layers in the head.

10. The method of claim 1 wherein the raw, fresh organ meat is preferably beef or pork's liver, heart, or brain.

11. The method of claim 1 wherein the raw, fresh organ meat may be replaced with herbs or spices.

12. The method of claim 1 wherein the raw, fresh organ meat may be replaced with a combination of different organ meat such as liver or brain and/or different herbs or spices.
